(19) United States
(12) Patent Application Publication
(10) Pub. No.: US 2025/0230395 A1
(43) Pub. Date: Jul. 17, 2025

(54) DEVICE FOR APPLYING STRETCHING STRESS TO CELLS

(71) Applicant: Beijing Chaoyang Hospital, Capital Medical University, China, Beijing (CN)

(72) Inventors: Yong HAI, Beijing (CN); Weishi LIANG, Beijing (CN); Xianjun QU, Beijing (CN); Bo HAN, Beijing (CN); Yeqiu XU, Beijing (CN); Duan SUN, Beijing (CN); Zhangfu LI, Beijing (CN); Yihan YANG, Beijing (CN); Peng YIN, Beijing (CN); Shuxiang CUI, Beijing (CN); Xinan ZHANG, Beijing (CN); Xinfeng WU, Beijing (CN)

(21) Appl. No.: 19/006,557

(22) Filed: Dec. 31, 2024

(30) Foreign Application Priority Data
Jan. 12, 2024 (CN) 202410052010.3

(51) Int. Cl. C12M 1/42; C12M 1/00; C12M 1/36

(52) U.S. Cl. CPC C12M 35/04; C12M 23/22; C12M 23/26; C12M 41/48

(57) ABSTRACT

A device for applying stretching stress to cells includes a culture plate. Cell holes are formed on the culture plate, a bottom of each of the cell holes is covered with an elastic membrane, adjacent two sides of the elastic membrane are fixedly connected with a bottom of the culture plate, and other adjacent two sides are each fixedly connected with a traction mechanism. The bottom of the cell hole is covered with the elastic membrane, cells adhere to the elastic membrane, the elastic membrane is deformed by traction mechanism.

### CROSS-REFERENCE TO RELATED APPLICATIONS

This application claims priority to Chinese Patent Application No. 202410052010.3, filed on Jan. 12, 2024, the contents of which are hereby incorporated by reference.

### TECHNICAL FIELD

The present disclosure relates to the technical field of experimental instruments, and in particular relates to a device for applying stretching stress to cells.

### BACKGROUND

It may be observed in clinical work and basic research that external stress stimulation has a significant impact on different kinds of cells in vivo or in vitro. In order to simulate the process of cells being stretched by external forces, cell stretching technology has emerged. Cell stretch may apply stress stimulation to cells cultured in vitro. Observing the changes after cell stretch is widely used in the fields of cell biology and biomedicine, for example, cell stretch may be used to study the basic biological processes such as cell migration, cell adhesion and cell apoptosis. Cell stretch is helpful to reveal the mechanism of invasion and metastasis of cancer cells and provide a new perspective and therapeutic target for cancer research. Studying the stretching force distribution of cells in three-dimensional environment may guide the design of tissue engineering and improve the biocompatibility and function of artificial tissues and organs. Cell stretch is also helpful to study the mechanical regulation mechanism of neuron growth and synapse formation. However, the existing cell stretch is operated in culture dishes, and there is no special device for providing stretching stress for cell culture. In order to better simulate the process of cell stretch, it is the most critical problem to apply an efficient device for applying stretching stress to cells.

### SUMMARY

An objective of the present disclosure is to provide a device for applying stretching stress to cells for solving the technical problems mentioned above.

The present disclosure provides a device for applying stretching stress to cells, which includes a culture plate, multiple cell holes are formed on the culture plate, a bottom of each of the cell holes is covered with an elastic membrane, adjacent two sides of the elastic membrane are fixedly connected with a bottom of the culture plate, and other adjacent two sides are each fixedly connected with a traction mechanism.

In an embodiment, the traction mechanism includes a slider fixedly connected with an edge of the elastic membrane and electric telescopic rods, and a side of the slider away from the elastic membrane is fixedly connected with a rod body of each of the electric telescopic rods.

In an embodiment, each slider corresponds to two electric telescopic rods, and the two electric telescopic rods are respectively located on two sides of the slider.

In an embodiment, a bottom of the slider is provided with a clip, the edge of the elastic membrane is located between the clip and the slider, and the clip is fixedly connected with the bottom of the slider through screws.

In an embodiment, a length of the slider is greater than or equal to a diameter of each of the cell holes.

In an embodiment, the elastic membrane is a silica gel membrane.

In an embodiment, the elastic membrane is square in a relaxed state.

In an embodiment, a cover plate is arranged above the cell holes, and the cover plate is made of transparent material.

In an embodiment, the device further includes a control system, the control system includes a programmable logic controller (PLC), a power supply and a control panel, and the PLC is electrically connected with the electric telescopic rods and the power supply, and the control panel is provided with a power key for controlling the power supply and working keys for controlling the electric telescopic rods.

In an embodiment, the working keys include selection keys for selecting a stretching area, a first knob for adjusting stretching force and a second knob for adjusting stretching time.

The present disclosure has following beneficial effects.

According to the present disclosure, the bottom of the cell hole is covered with the elastic membrane, the cells adhere to the elastic membrane, and the adjacent two sides of the elastic membrane are fixedly connected to the bottom of the culture plate, and the other adjacent two sides are each fixedly connected with the traction mechanism. After the cells adhere to the wall of the elastic membrane, the elastic membrane is deformed via the pulling of the traction mechanism, and the cells adhered to the elastic membrane are arranged in polarity in the traction direction, and the cells are elongated to achieve the purpose of cell traction. By controlling the traction mechanism, the device may ensure the consistency of cell stretching degree, and the device for applying stretching stress to cells has the advantages of being easy to adjust and accurate in quantification.

### BRIEF DESCRIPTION OF THE DRAWINGS

In order to explain the embodiments of the present disclosure or the technical solution in the prior art more clearly, the drawings needed in the embodiments will be briefly introduced below. Apparently, the drawings in the following description are only some embodiments of the present disclosure. For one of ordinary skill in the art, other drawings may be obtained according to these drawings without paying creative labor:

FIG. 1 is a schematic diagram of an overall structure according to the present disclosure.

FIG. 2 is a schematic diagram of a three-dimensional structure of a single cell hole and a traction mechanism in the present disclosure.

FIG. 3 is a schematic side view of the single cell hole and the traction mechanism in the present disclosure.

FIG. 4 is a schematic structural diagram of an elastic membrane and a slider in the present disclosure.

FIG. 5 is a block diagram of a control system in the present disclosure.

### DETAILED DESCRIPTION OF THE EMBODIMENTS

In the following, the technical solutions in the embodiments of the present disclosure will be clearly and completely described with reference to the attached drawings. Apparently, the described embodiments are only a part of the embodiments of the present disclosure, but not all the embodiments. Based on the embodiments in the present disclosure, all other embodiments obtained by one of ordinary skill in the art without creative effort belong to the protection scope of the present disclosure.

In the description of the present disclosure, it should be understood that the terms “center”, “longitudinal”, “transverse”, “length”, “width”, “thickness”, “up”, “down”, “front”, “back”, “left”, “right”, “vertical”, “horizontal”, “top”, “bottom”, “inside”, “outside”, “clockwise”, “counterclockwise”, etc. indicate orientation or positional relationships based on the orientation or positional relationships shown in the accompanying drawings, only for the convenience of describing the present disclosure, and do not indicate or imply that the device or element referred to must have a specific orientation, be constructed and operated in a specific orientation, and therefore cannot be understood as a limitation of the present disclosure.

In addition, the terms “first” and “second” are only used for descriptive purposes, and cannot be understood as indicating or implying relative importance or implicitly indicating the number of indicated technical features. Therefore, the features defined as “first” and “second” may explicitly or implicitly include one or more of said features. In the description of the present disclosure, “multiple” means two or more, unless otherwise specifically defined. In addition, the terms “install”, “connect to” and “connect with” should be understood broadly, for example, the terms may be understood as a fixed connection, a detachable connection, or an integrated connection; the terms may be understood as a mechanical connection or an electrical connection; and the terms may be understood as a direct connection, or an indirect connection through an intermediate medium, which may be the internal connection between two components. For those skilled in the art, the specific meanings of the above terms in the present disclosure may be understood in specific situations.

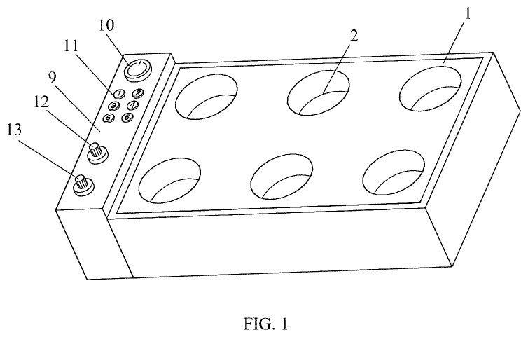

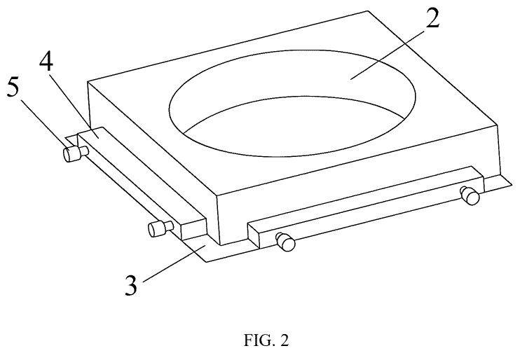

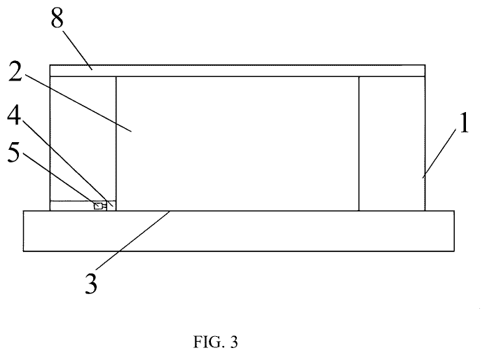

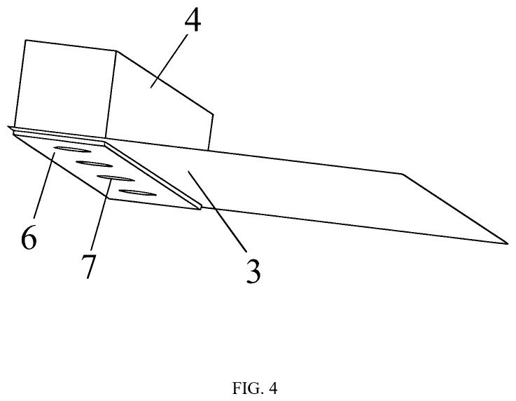

In an embodiment, a device for applying stretching stress to cells is provided, as shown in FIG. 1 to FIG. 4, which includes a culture plate 1, multiple cell holes 2 are formed on the culture plate 1, and the bottom of each cell hole 2 is covered with an elastic membrane 3, and the adjacent two sides of the elastic membrane 3 are fixedly connected with the bottom of the culture plate 1 (which may be fixed by bonding), and the other adjacent two sides are each fixedly connected with a traction mechanism.

The traction mechanism includes a slider 4 and electric telescopic rods 5, the slider 4 is fixedly connected to the edge of the elastic membrane 3. The side of the slider 4 away from the elastic membrane is fixedly connected with the rod body of the electric telescopic rod 5. Specifically, the bottom of the slider 4 is provided with a clip 6, and the edge of the elastic membrane 3 is located between the clip 6 and the slider 4, and the clip 6 is fixedly connected with the bottom of the slider 4 through screws 7. The bottom of the slider 4 is provided with grooves corresponding to screw heads to prevent the screw heads from protruding from the lower surface of the slider. Each slider 4 corresponds to two electric telescopic rods 5, and the electric telescopic rods 5 are respectively located on two sides of the slider 4.

The length of the slider 4 is greater than or equal to the diameter of the cell hole 2, and the elastic membrane 3 is a silica gel membrane. The silica gel membrane is a medical silica gel membrane for the clinical skin and soft tissue expander. The elastic membrane is square in a relaxed state. In addition, a cover plate 8 made of transparent material is arranged above the cell holes 2.

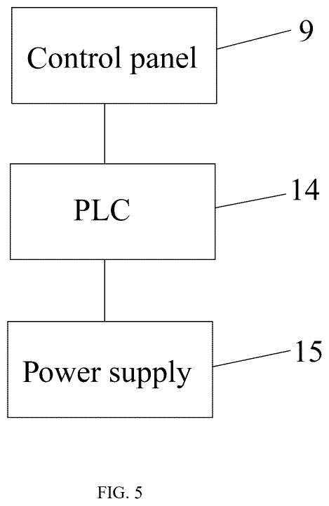

The device further includes a control system, as shown in FIG. 5, and the control system includes a programmable logic controller (PLC) 14, a power supply 15 and a control panel 9. The PLC is electrically connected with the electric telescopic rods and the power supply respectively, and the control panel 9 is provided with a power key 10 for controlling the power supply and working keys for controlling the electric telescopic rods. The working keys include selection keys 11 for selecting the stretching area, a knob 12 for adjusting stretching force and a knob 13 for adjusting stretching time. The above-mentioned part about the control system, in which the electrical connection relationship and working principle of electrical components are applicable to the prior art, will not be repeated here.

Working and using process are as follows.

Cells are inoculated into the cell holes 2, covering with the cover plate 8, and the device is put in a cell incubator for a certain period of time. After observing the cell adherent growth through an optical microscope, the following operations are performed, so that the cells are in a state of being stimulated by stretching stress.

Specifically, when in use, the number of the cell hole is first selected, and then the displacements of the electric telescopic rods 5 corresponding to the selected cell hole may be controlled by the knob 12 for adjusting stretching force, so that the cells are subjected to traction, and the whole traction process may be observed by a microscope during the traction. The cells attached to the elastic membrane 3 will be arranged in polarity in the traction direction, and the cells can be elongated to achieve the objective of cell traction. In addition, the duration of cell stretching is set by rotating the knob 13 for adjusting stretching time, so that the traction may be accurately controlled, and the cell traction may achieve the effect for using. The traction cells may be effectively observed by using the microscope, so as to ensure the cell traction effect.

According to the present disclosure, traction mechanisms are arranged in both the X direction and the Y direction of the elastic membrane 3 (that is, two mutually perpendicular directions in the same plane), so that simultaneous traction in both directions may be realized, so that the mechanical traction force of the electric telescopic rods 5 may be transmitted to cell through the elastic membrane.

Finally, it should be explained that the above embodiments are only used to illustrate the technical solution of the present disclosure, but not to limit it. Although the present disclosure has been described in detail with reference to the foregoing embodiments, it should be understood by those skilled in the art that the technical solution described in the foregoing embodiments may still be modified, or some or all of its technical features may be replaced by equivalents. However, these modifications or substitutions do not make the essence of the corresponding technical solutions deviate from the scope of the technical solutions of various embodiments of the present disclosure.

What is claimed is:

1. A device for applying stretching stress to cells, comprising:
a culture plate,
wherein a plurality of cell holes are formed on the culture plate, a bottom of each of the cell holes is covered with an elastic membrane, adjacent two sides of the elastic membrane are fixedly connected with a bottom of the culture plate, and other adjacent two sides are each fixedly connected with a traction mechanism.

2. The device for applying stretching stress to cells according to claim 1, wherein the traction mechanism comprises a slider fixedly connected with an edge of the elastic membrane and electric telescopic rods, and a side of the slider away from the elastic membrane is fixedly connected with a rod body of each of the electric telescopic rods.

3. The device for applying stretching stress to cells according to claim 2, wherein each slider corresponds to two electric telescopic rods, and the two electric telescopic rods are respectively located on two sides of the slider.

4. The device for applying stretching stress to cells according to claim 3, wherein a bottom of the slider is provided with a clip, the edge of the elastic membrane is located between the clip and the slider, and the clip is fixedly connected with the bottom of the slider through screws.

5. The device for applying stretching stress to cells according to claim 4, wherein a length of the slider is greater than or equal to a diameter of each of the cell holes.

6. The device for applying stretching stress to cells according to claim 1, wherein the elastic membrane is a silica gel membrane.

7. The device for applying stretching stress to cells according to claim 6, wherein the elastic membrane is square in a relaxed state.

8. The device for applying stretching stress to cells according to claim 1, wherein a cover plate is arranged above the cell holes, and the cover plate is made of transparent material.

9. The device for applying stretching stress to cells according to claim 3, further comprising a control system, wherein the control system comprises a programmable logic controller (PLC), a power supply and a control panel, wherein the PLC is electrically connected with the electric telescopic rods and the power supply, and the control panel is provided with a power key for controlling the power supply and working keys for controlling the electric telescopic rods.

10. The device for applying stretching stress to cells according to claim 9, wherein the working keys comprise selection keys for selecting a stretching area, a first knob for adjusting stretching force and a second knob for adjusting stretching time.
